(19) United States
(12) Patent Application Publication
(10) Pub. No.: US 2013/0133407 A1
(43) Pub. Date: May 30, 2013

(54) Methods For Analysis Of Water And Substrates Rinsed In Water

(71) Applicant: MEMC Electronic Materials, Inc., St. Peters, MO (US)

(72) Inventor: Larry Wayne Shive, St. Charles, MO (US)

(73) Assignee: MEMC ELECTRONIC MATERIALS, INC., St. Peters, MO (US)

(21) Appl. No.: 13/750,371

(22) Filed: Jan. 25, 2013

Related U.S. Application Data
(62) Division of application No. 12/967,382, filed on Dec. 14, 2010.
(60) Provisional application No. 61/289,864, filed on Dec. 23, 2009.

(51) Int. Cl. G01N 33/18

(52) U.S. Cl. CPC G01N 33/18
USPC 73/61.72

(57) ABSTRACT

A method is disclosed for determining metal content in a container of water. The method includes contacting a substrate with the water for a predetermined period of time. The substrate is then dried and analyzed to determine the metal content of the substrate surface. A determination is then made of the metal content in the water from the metal content on the substrate surface.

### CROSS-REFERENCE TO RELATED APPLICATION

This application is a divisional of U.S. patent application Ser. No. 12/967,382 filed Dec. 14, 2010, which claims the benefit of U.S. Provisional Patent Application No. 61/289,864 filed Dec. 23, 2009, the entire disclosures of which are hereby incorporated by reference.

### BACKGROUND

Impurities present on the surface of a substrate, such as a semiconductor wafer, may negatively affect the material properties of the substrate. Some impurities may be deposited on the surface of the substrate by water used to rinse the substrate. Accordingly, it is desirable to reduce or eliminate the amount of impurities contained in the water in which the wafers are rinsed. Water used to rinse the substrate is often analyzed to determine the amount and type of impurities present therein so that the proper filters or other remediation systems may be selected and used to reduce or eliminate the impurities contained in the water.

In known systems, the amount and type of impurities deposited on the surface of the substrate is determined by various analytical methods. The analytical methods are capable of determining the presence and amount of impurities above a set threshold level. However, impurities deposited on the surface of the substrate below the threshold level may negatively affect the properties of the substrate or components formed from the substrate. Prior systems are thus incapable of detecting impurities deposited on the surface of substrates that may negatively affect the properties of the substrate.

### BRIEF SUMMARY

A first aspect is directed to a method for determining metal content in a container of water. The method comprises contacting a substrate with the water for a predetermined period of time. The substrate is then dried and analyzed to determine the metal content of the substrate surface. A determination is then made of the metal content in the water from the metal content on the substrate surface.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

FIG. 1 is a partially schematic cross-section of a tank for rinsing a wafer in accordance with one embodiment;

FIG. 2 is a schematic of a system for supplying water to the tank shown in FIGS. 1 and 5 and receiving water from the tank in a reservoir.

FIG. 3 is a top plan view of an exemplary wafer;

FIG. 4 is a top plan view of another exemplary wafer;

FIG. 5 is a partially schematic cross-section of a tank for rinsing a wafer in accordance with another embodiment;

FIG. 6 is a flow diagram showing a method of predicting the amount of contaminants deposited on the surface of a wafer;

FIG. 7 is a flow diagram showing another method of predicting the amount of contaminants deposited on the surface of a wafer;

FIG. 8 is a flow diagram showing a method of predicting the amount of contaminants deposited on the surface of a substrate; and

FIGS. 9-11 depict experimental data in the form of graphs showing the relationship between the density of various contaminants deposited on the surface of the wafer W at different predetermined amounts of time.

### DETAILED DESCRIPTION

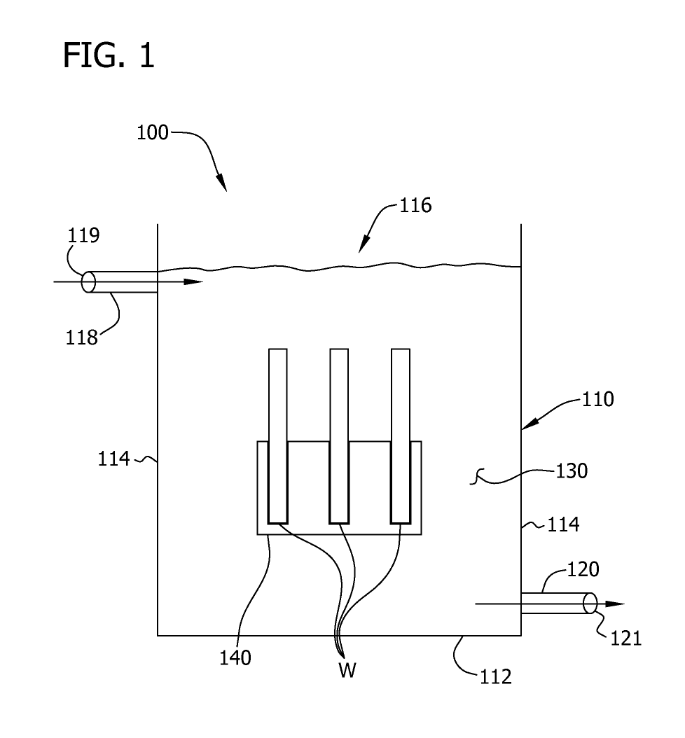

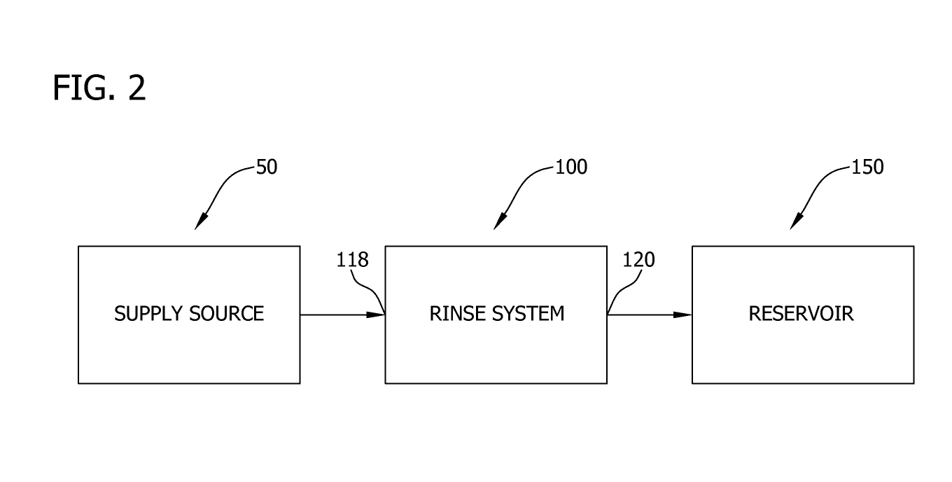

Referring initially to FIGS. 1 and 2, a system for rinsing a wafer W (broadly, a substrate) is generally referred to as 100. Reference is made herein to contamination deposited on the surface of the wafer W. The amount of contaminants deposited on the surface of the wafer W may be expressed as a concentration of contaminants (i.e., atoms of contaminants per unit area), as parts-per notation (i.e., parts per million or trillion), or as mass per unit area (i.e., grams per mm²).

FIG. 2 is a schematic diagram showing a supply source 50, the system 100, and a reservoir 150 (or drain reservoir). Water (i.e., a fluid) is supplied to the system 100 by the supply source 50. The supply source 50 may include one or more wells or a supply of water (e.g., a municipal water supply system). The supply source 50 may include one or more treatment or filtering mechanisms to filter impurities (e.g., particles and metals) from the water prior to supplying it to the system 100. The system 100 and supply source 50 are coupled together through any suitable fluid connection mechanism (e.g., hoses and/or pipes). In one embodiment, supply source 50 includes one or more pumps to pump water from the supply source to the system 100. In some embodiments, the system 100 only samples a portion of the water supplied by the supply source 50. In other embodiments, the system 100 samples all of the water supplied by the supply source 50. The supply source 50 may thus supply a continuous flow of water to the system 100 such that water enters the system 100 and flows therethrough to the reservoir 150. After receiving the water from the system 100, the reservoir 150 may dispose of the water. In other embodiments, the reservoir 150 may store or supply the water to another process for further use or treatment.

Referring now to FIG. 1, the system 100 includes a tank 110 (broadly, a “container”) which has a bottom member 112 and four vertical side members 114 coupled thereto. The bottom member 112 and side members 116 may be formed from any suitable material, such as metal or plastic. Moreover, while the tank 110 of FIG. 1 is rectangular in shape, in other embodiments the tank may be shaped differently.

The bottom member 112 and side members 114 form a water-tight enclosure that is open at its top 116. The members 112, 114 are joined together with any suitable joining mechanism, such as welding or adhesive bonding. Moreover, in one embodiment, the members 112, 114 are integrally formed from the same blank of material such that joining mechanisms are unnecessary. In other embodiments, the tank 110 includes an additional top member (not shown) coupled to side members 114 such that the tank is an enclosed, multi-sided structure.

A liquid 130 is disposed within the tank 110. The amount of liquid 130 in the tank 110 is great enough such that the wafers W are completely submerged in the liquid 130. However, in one embodiment the wafers W are not completely submerged in the liquid 130. The liquid 130 in this embodiment is water. In other embodiments, the liquid 130 is any suitable liquid (e.g., a solvent) that has sufficient viscosity to flow through the tank 110.

The tank 110 of this embodiment has an inlet 118 and an outlet 120 (or drain) to permit the flow therethrough of the fluid 130. The inlet 118 is coupled to the supply source 50 and the outlet 120 is coupled to reservoir 150. In the embodiment of FIG. 1, the inlet 118 and outlet 120 are tubes with fittings (not shown) disposed on their respective outer ends 119, 121. The fittings permit coupling of the inlet 118 and outlet 120 to other fluid-flow mechanisms (e.g., pipes, hoses, or tubes) that in turn are coupled to the supply source 50 and reservoir 150, respectively.

The cross-sectional areas of the inlet 118 and outlet 120 are sufficiently sized to achieve a desired flow rate through the tank 110. In the exemplary embodiment, the cross-sectional areas of the inlet 118 and outlet 120 are sized such that the flow rate is between 0 liters per minute and 50 liters per minute. The positions of the inlet 118 and outlet 120 shown in FIG. 1 are exemplary, and the inlet 118 and outlet 120 may be in different positions. For example, either or both of the inlet 118 and outlet 120 may be positioned adjacent the top 116 or bottom member 112 of the tank 110 without departing from the scope of the embodiments. Moreover, in some embodiments the outlet 120 is configured and positioned such that liquid 130 overflows from the tank through the outlet 120. In these embodiments, the outlet 130 is similar in function and configuration to a spillway.

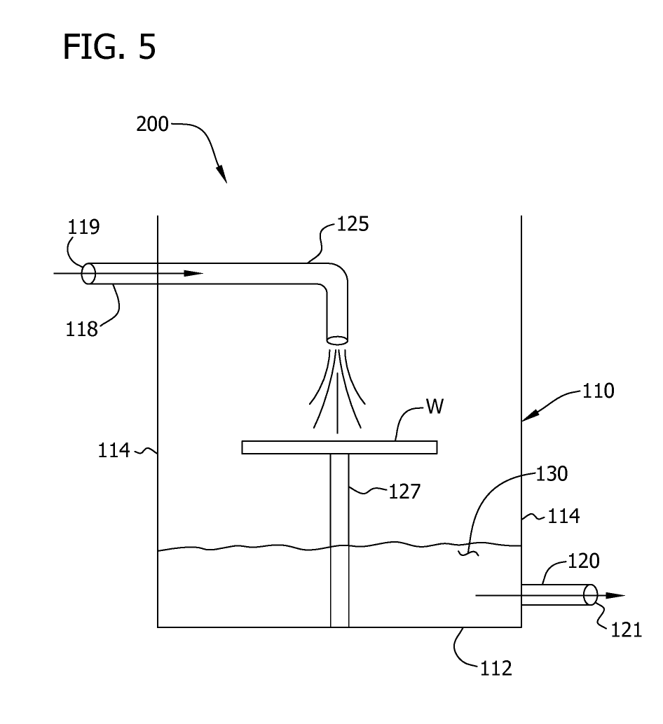

In FIG. 1, the wafers W are positioned in the interior of the tank 110 by a suitable support structure 140. The support structure 140 is configured to permit the free flow of liquid 130 around substantially the entire outer surfaces of the wafer W. The support structure 140 is formed from a material that is non-reactive with the liquid 130 and does not release contaminants when in the presence of the liquid and may be coated with non-reactive layer of material (e.g., Teflon®). In the embodiment of FIG. 1, three wafers W are positioned in the tank 110 by the support structure 140, while in other embodiments more or fewer wafers W are positioned in the tank. Moreover, while the wafers W are shown in FIG. 1 as being positioned by the support structure 140 in a substantially vertical arrangement, the wafers W may instead be supported in a different orientation (e.g., horizontal or angled with respect to the bottom member 112 as shown in FIGS. 1 and 5) without departing from the scope of the disclosure. In some embodiments, the support structure 140 is integrally formed with the tank 110, while in others the support structure is a separate component that is placed within the tank 110. Wafers W may be placed into the support structure 140 either before or after the tank 110 is filled with liquid 130. In embodiments where the tank 110 is empty and contains no liquid, the wafers W may be positioned therein by a vacuum wand, such as a wand having a tip made of Teflon or other suitable material. In embodiments where the tank 110 is substantially full of liquid 130, the wafers may be positioned therein by robotic effectors coated in Teflon or Teflon-like material.

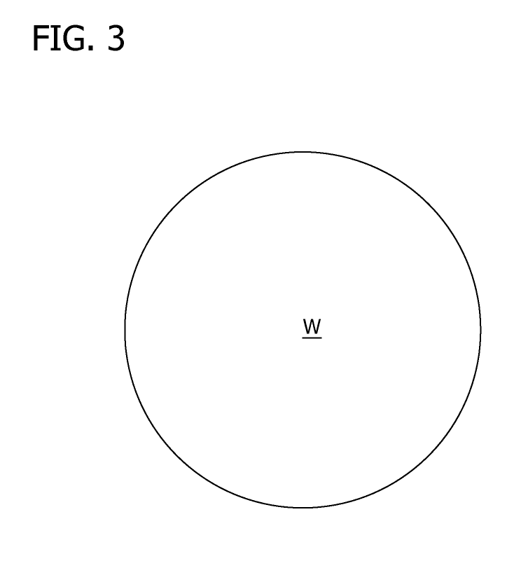

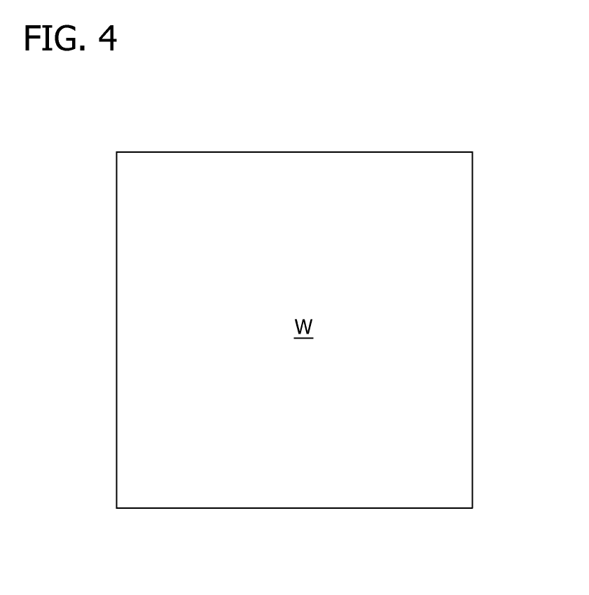

Referring now to FIGS. 3 and 4, two differently shaped wafers are illustrated. In the embodiment of FIG. 3, the wafer W is sliced from an ingot, as is customary in the industry, and can be made from silicon, germanium, gallium arsenide, or other suitable materials. Alternatively, the wafer W may be square or rectangular as seen in FIG. 4 (e.g., of the type commonly used in the manufacture of solar cells). In other embodiments, different types of substrates may be rinsed in the system 100. The substrate may be of any type that has a surface onto which impurities may be deposited.

Referring now to FIG. 5, a system 200 is shown that is similar to the system 100 of FIG. 1 and like reference numerals are used to refer to similar components. The system 200 generally differs from the system 100 in that the wafer W is positioned in a different configuration and is not submerged or immersed in the liquid 130. The wafer W in the system 200 is positioned in a generally horizontal orientation and supported by a support member 127. The support member 127 supports and positions the wafer W vertically above the surface of the liquid 130 in the tank 110. In some embodiments, the support member 127 is coupled to a rotary motion device (e.g., a motor) such that the support member (and hence the wafer W positioned thereon) is selectively rotatable. According to one embodiment, the support member 127 and wafer W positioned thereon are rotatable from 0 RPM (i.e., stationary) to 2000 RPM.

The inlet 118 of the tank 110 has an extension 125 attached thereto and configured to direct the flow of liquid onto approximately a geometric center of the surface of the wafer W. The extension 125 thus directs the flow of liquid 130 to contact the surface of the wafer W. After contacting the surface of the wafer W, the liquid 130 flows off of the surface of the wafer W and is collected in the tank 110 before being directed therefrom through the outlet 120. Moreover, reduced liquid flow rates may be used in the system 200 compared to those used in the system 100. For example, after the surface of the wafer W is sufficiently wetted with liquid 130, the flow rate may be between 0 liters per minute and 2 liters per minute.

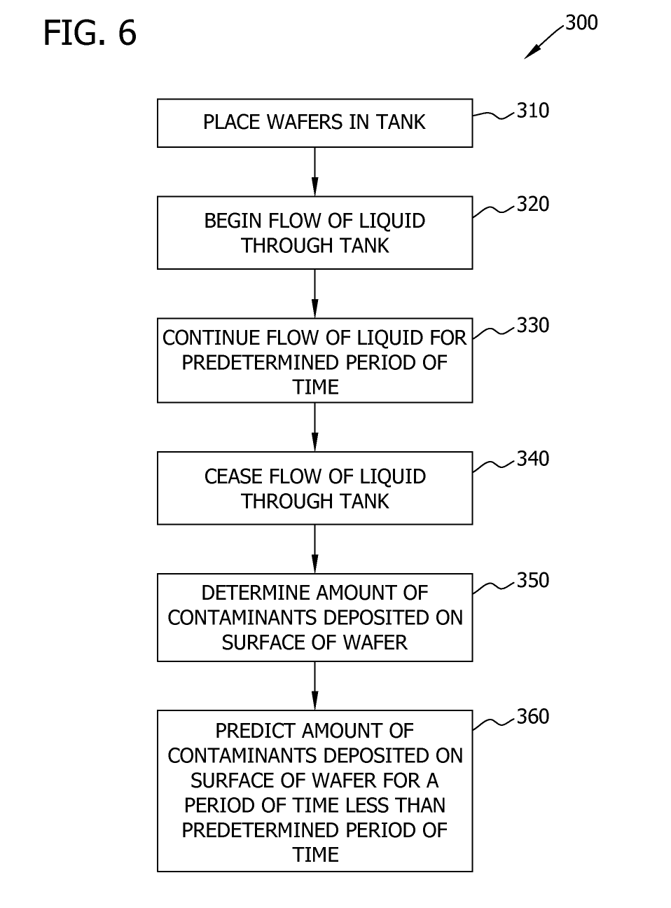

FIG. 6 is a flow diagram depicting a method 300 of predicting the amount of contaminants deposited on the surface of a wafer. In the method 300, the wafer W is contacted by liquid by immersing the wafer in liquid 130. In known systems, a wafer is typically rinsed in a tank for a period of time (e.g., 1 to 10 minutes). Contaminants present in the liquid used to rinse the wafer (e.g., water) are often deposited on the surface of the wafer while the wafer is rinsed in the tank. As described above, amounts of contaminants deposited on the surface of a wafer during a rinse may be such that, while they negatively affect the properties of the wafer W, they are not detectable by known detection systems (e.g., inductively coupled plasma mass spectrometry). One such contaminant is nickel, the presence of which on the surface of the wafer W negatively affects the properties of the wafer even when the amount of nickel deposited thereon is below the level detectable by known systems. Other contaminants that may be deposited include sodium, aluminum, calcium, titanium, chromium, iron, cobalt, copper, and zinc. Other metal contaminants may also affect the material properties of the wafer W, even though the presence of metal contaminants under a certain concentration is not detectable by known systems.

As described herein, the method 300 permits the detection of relatively small amounts of contamination present in the liquid 130 that are not otherwise detectable with known systems. For example, known systems are generally only able to detect concentrations of contaminants on the surface of the wafer W that are greater than about 2e8 atoms/cm². The method 300 described below is capable of detecting concentrations of contaminants that are substantially below 2e8 atoms/cm². For example, the method 300 is capable of detecting concentrations of contaminants in the range of 1e5 atoms/cm².

The method 300 of this embodiment begins at block 310 with the placing of wafers W in the tank 110. The tank 110 may be cleaned prior to the placement of the wafers W therein to ensure that the tank is free from contamination. In some embodiments, the tank 110 may be washed with acid. The wafers W are placed in the tank 110 and positioned therein by the wafer support. While reference is made herein to a plurality of wafers W being placed in the tank 110, a single wafer may instead be placed in the tank. The placement of multiple wafers W in the tank 110 results in a correspondingly larger sample of data collected in accordance with the method 300.

In block 320, the flow of liquid 130 through the tank 110 begins. The liquid 130 in this embodiment is water. In other embodiments the liquid 130 may be any suitable liquid, such as a solvent. The liquid 130 first flows into the tank 110 through the inlet 118. The liquid 130 may first be filtered before entering the tank 110 through the inlet 118. In one embodiment, the liquid 130 (e.g., water) may be filtered such that it has a low enough level of contaminants and is referred to as ultra pure water (i.e., water containing less 1 part-per-trillion (ppt) of any metal contaminant). As the liquid 130 flows into the tank 110, the level of the liquid rises and eventually reaches the level of the outlet 120 of the tank. The liquid 130 then flows out of the tank 110 through the outlet 120. The liquid 130 may then be disposed of or recycled after flowing out of the tank 110. As described above, the wafers W may instead be placed within the tank 110 after it is filled with liquid 130.

At block 330, the flow of liquid 130 through the tank 110 continues for a predetermined period of time. In some embodiments, the predetermined period of time is referred to as a soak time. The predetermined period of time may be selected according to numerous factors. For example, if a threshold for the detection of a contaminant on the surface of the wafer W is 2e8 atoms/cm², the predetermined period of time may be selected such that the amount of contamination deposited on the surface of the wafer W is likely to exceed the threshold detection level. According to some embodiments, the predetermined period of time is approximately 100 to 150 times greater than the normal rinse time. With a rinse time of five minutes, the predetermined period of time is thus in the range of 500 to 1000 minutes. In one embodiment, the predetermined period of time is 750 minutes.

The flow of liquid 130 through the tank 110 ceases at block 340. After cessation of the flow of liquid 130, the liquid may be drained or otherwise removed from the tank. The wafers W may then be dried such that any residual liquid 130 present on the surface is removed. In another embodiment, the wafers W may be removed from the tank 110 while liquid is still present therein such that the wafers are at least partially immersed in the liquid prior to their removal. The wafers W may be removed from the tank 110 in this embodiment with the same type of robotic mechanism described above.

In block 350 the amount of contaminants deposited on the surface of the wafer W are determined. Various methods may be used to determine the amount and/or concentration of the contaminants deposited on the surface of the wafer W. For example, inductively coupled plasma mass spectrometry (ICP-MS) may be used to analyze the surface of the wafer W to determine the amount and/or concentration of the contaminants deposited thereon during the method 300. Concentration of contaminants may be expressed as the number of atoms of contaminants deposited on a given area of the surface of the wafer (e.g., atoms per cm². In other embodiments, different methods may be used to determine the amount and/or concentration of the contaminants deposited on the surface of the wafer W, such as total reflectance X-ray fluorescence (TXRF).

At block 360, a prediction is made as to the amount of contaminants deposited on the surface of the wafer W for a period of time less than the predetermined period of time in block 330. The period of time less than the predetermined period of time in one embodiment is the typical rinse time for the wafer W (e.g., 1 to 10 minutes). In one embodiment, the typical rinse time is 5 minutes and the predetermined period of time is 750 minutes.

In some embodiments, the steps performed in blocks 310-350 may only be performed either to establish a base line level of contamination or to verify the expected contamination levels. Once the determination is made in block 350, the prediction made in block 360 may be performed independently each time a wafer is rinsed in the liquid. Thus, the determination made in block 350 is not required to be performed every time the prediction in block 360 is performed. Instead, the steps performed in block 310-350 may be performed to calibrate the rinsing system, and the prediction performed in block 360 is performed on each wafer rinsed in the rinsing system.

The rate of deposition of contamination on the surface of the wafer W is assumed to be generally linear, and as such a linear interpolation is used to predict or determine the amount and/or concentration of contaminants that are deposited on the surface of the wafer during the typical wafer rinse time. For example, in one embodiment 2e10 atoms/cm² were deposited on the surface of the wafer in 750 minutes and a rinse time of the wafers W is 5 minutes. The concentration of contamination deposited on the surface of the wafer W is thus determined by multiplying the concentration of contaminants determined in block 350 by the ratio of the typical rinse time (e.g., 5 minutes) to the predetermined period of time (e.g., 750 minutes). In this embodiment, the concentration of the contaminants deposited on the surface of the wafer during a typical rinse is thus determined to be 1.33e8 atoms/cm². Accordingly, the linear interpolation is thus represented by the equation:

$$c = \frac{Tr}{Tp} * Ec ,$$

where c equals the concentration of contaminants deposited on the surface of the wafer during a typical rinse of the wafer W, Tr equals the length of time of a typical wafer rinse, Tp equals the predetermined period of time, and Ec equals the concentration of contaminants determined in block 350.

In other embodiments, the rate of deposition of contamination on the surface W is not generally linear. In these embodiments, the method 300 may be repeated several times and each time the predetermined period of time may be varied. Accordingly, multiple pairs of values for contaminant concentration levels and corresponding predetermined periods of time are determined. The pairs of values may then be used in any number of numerical interpolation methods to determine the rate of deposition of contaminants on the surface of the wafer W. The determined rate of deposition may then be multiplied by the rinse time of the wafer to arrive at the amount and/or concentration of contaminants deposited on the surface of the wafer.

The method 300 described above thus permits the detection of amounts of contaminants in the liquid 130 well below those detectable by known systems. In known systems, the lower limit of detection of the most sensitive ICP-MS methods is about 0.1 ppt. Accordingly, the presence of contaminants in the liquid 130 and on the surface of the wafer W are detectable by the method 300 even though the amount of contaminants is well below those detectable by known systems.

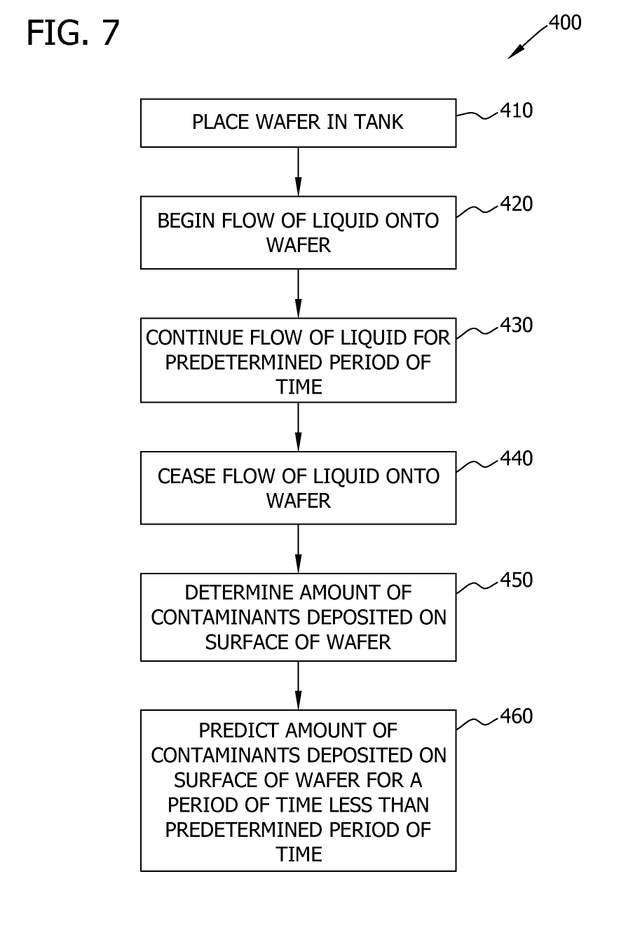

FIG. 7 is a flow diagram depicting a method 400 of predicting the amount of contaminants deposited on the surface of a wafer. In the method 400, the wafer W is contacted by liquid by directing the flow of liquid 130 to contact the surface of the wafer W. The method 400 is generally similar to the method 300 described above, except that liquid is directed to flow onto the wafer. In some embodiments, the method 400 is used in conjunction with the system 100 or system 200 described above. The method 400 of this embodiment begins at block 410 with the placing of the wafer W in the tank 110. The tank 110 may be cleaned prior to the placement of the wafers W therein to ensure that the tank is free from contamination. In some embodiments, the tank 110 may be washed with acid. The wafer W is placed in the tank 110 and positioned therein by the support member 127.

In block 420, the flow of liquid 130 onto the surface of the wafer W begins. The liquid 130 in this embodiment is water. In other embodiments, the liquid 130 may be any suitable liquid, such as a solvent. The liquid 130 first flows into the tank 110 through the inlet 118. The liquid 130 may first be filtered before entering the tank 110 through the inlet 118. In one embodiment, the liquid 130 (e.g., water) may be filtered such that it has a low enough level of contaminants and is referred to as ultra pure water. The liquid 130 may be directed to flow onto the surface of the wafer W by the extension 125 coupled to the inlet 118. After flowing across the surface of the wafer W, the liquid 130 then flows into the tank 110. The liquid 130 then flows out from the tank 110 through the outlet 120. The liquid 130 may then be disposed of or recycled after flowing out of the tank 110. In another embodiment, the wafer W may be rotated by the support member 127 as liquid flows onto the surface of the wafer W,

At block 430, the flow of liquid 130 onto the surface of the wafer W continues for a predetermined period of time. For example, if a threshold for the detection of a contaminant on the surface of the wafer W is 2e8 atoms/cm², the predetermined period of time may be selected such that the amount of contamination deposited on the surface of the wafer W is likely to exceed the threshold detection level. According to some embodiments, the predetermined period of time is approximately 100 to 150 times greater than the normal rinse time. With a rinse time of five minutes, the predetermined period of time is thus in the range of 500 to 1000 minutes. In one embodiment, the predetermined period of time is 750 minutes.

The flow of liquid 130 onto the surface of the wafer W ceases at block 440. After cessation of the flow of liquid 130, the liquid may be drained or otherwise removed from the tank. The wafer W may then be dried such that any residual liquid 130 present on the surface is removed.

In block 450 the amount of contaminants deposited on the surface of the wafer W are determined in a manner similar to or the same as that described above in block 350. At block 460, a prediction is made as to the amount of contaminants deposited on the surface of the wafer W for a period of time less than the predetermined period of time in block 430. The period of time less than the predetermined period of time in one embodiment is the typical rinse time for the wafer W (e.g., 1 to 10 minutes). In one embodiment, the typical rinse time is 5 minutes and the predetermined period of time is 750 minutes. The prediction made in block 460 is done in a substantially similar or the same method as that described above in block 360.

The method 400 described above thus permits the detection of amounts of contaminants in the liquid 130 well below those detectable by known systems. Accordingly, the presence of contaminants in the liquid 130 and on the surface of the wafer W is detectable even though the amount of contaminants is well below those detectable by known systems.

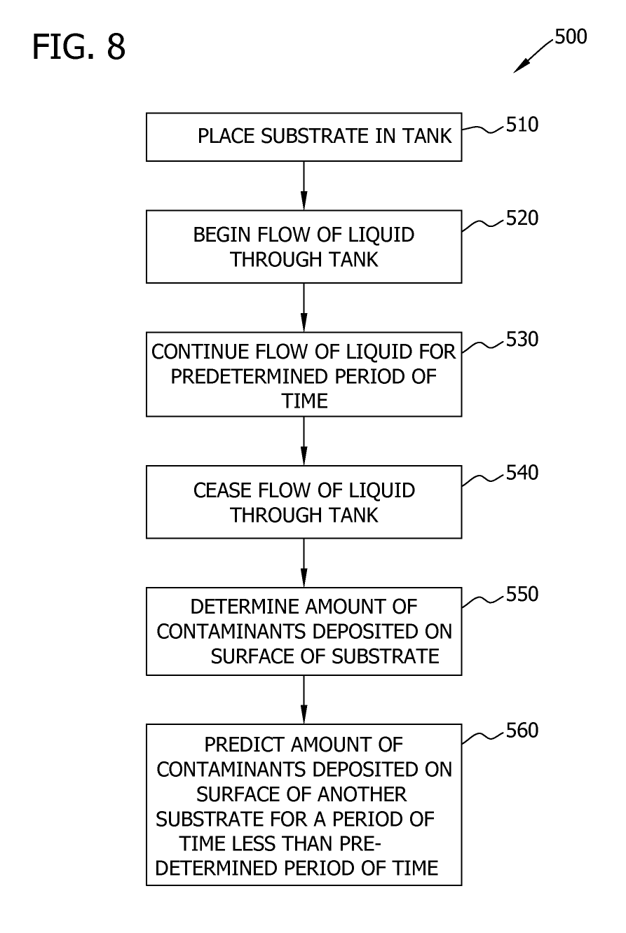

FIG. 8 is a flow diagram depicting a method 500 of predicting the amount of contaminants deposited on the surface of a substrate. The method 500 is generally similar to the method 300 described above, except that the method 500 is used to predict the contaminants deposited on the surface of a substrate, instead of a wafer. The method 500 uses the amount of contaminants deposited on the surface of the wafer W to predict the amount of contaminants deposited on the surface of the substrate. The method 500 is useful in predicting the amount of contaminants deposited on substrates that have material properties which make them ill-suited for typical contaminant testing methods (e.g., ICP-MS) because of the testing methods use of acid or other chemicals. Examples of such substrates include those comprising quartz, sapphire, germanium, or any other material that is ill-suited for typical contaminant testing methods. While a wafer is used to predict the amount of contaminants deposited on the surface of a substrate in this embodiment, another substrate may be used instead of the wafer for this purpose. Moreover, the method 500 is also used to determine the metals content of water in which the wafer W is placed.

Although the method 500 is described herein for use with the system 100, the method may be used in conjunction with either the system 100 or system 200 described above and thus the wafer W may either be submerged in the liquid 130 or its surface may instead be contacted by a flow of liquid 130.

The method 500 of this embodiment begins at block 510 with the placing of the wafer W in the tank 110. The wafer W may be placed in the tank by a suitable vacuum wand as described above. The tank 110 may be cleaned prior to the placement of the wafer W therein to ensure that the tank is free from contamination. In some embodiments, the tank 110 may be washed with acid. The wafer W is placed in the support member 140 in the tank 110.

In block 520, the flow of liquid 130 onto the surface of the wafer W begins. The liquid 130 in this embodiment is water. In other embodiments the liquid 130 may be any suitable liquid, such as a solvent. The liquid 130 first flows into the tank 110 through the inlet 118. The liquid 130 may first be filtered before entering the tank 110 through the inlet 118. In one embodiment, the liquid 130 (e.g., water) may be filtered such that it has a low enough level of contaminants and is referred to as ultra pure water. The liquid 130 then flows out from the tank 110 through the outlet 120. The liquid 130 may then be disposed of or recycled after flowing out of the tank 110.

At block 530, the flow of liquid 130 into the tank 110 continues for a predetermined period of time. For example, if a threshold for the detection of a contaminant on the surface of the wafer W is 2e8 atoms/cm², the predetermined period of time may be selected such that the amount of contamination deposited on the surface of the wafer W is likely to exceed the threshold detection level. According to some embodiments, the predetermined period of time is approximately 100 to 150 times greater than the normal rinse time. With a rinse time of five minutes, the predetermined period of time is thus in the range of 500 to 1000 minutes. In one embodiment, the predetermined period of time is 750 minutes.

The flow of liquid 130 into the tank 110 ceases at block 540. After cessation of the flow of liquid 130, the liquid may be drained or otherwise removed from the tank 110. The wafer W may then be dried such that any residual liquid 130 present on the surface is removed.

In block 550 the amount of contaminants deposited on the surface of the wafer W are determined in a manner similar to or the same as that described above in block 350 or block 450. At block 560, a prediction is made as to the amount of contaminants deposited on the surface of a substrate for a period of time less than the predetermined period of time in block 530. The period of time less than the predetermined period of time in one embodiment is the typical rinse time for the substrate (e.g., 1 to 10 minutes). In one embodiment, the typical rinse time is 5 minutes and the predetermined period of time is 750 minutes. The prediction made in block 560 is done in a substantially similar or the same method as that described above in block 360.

### Experimental Data

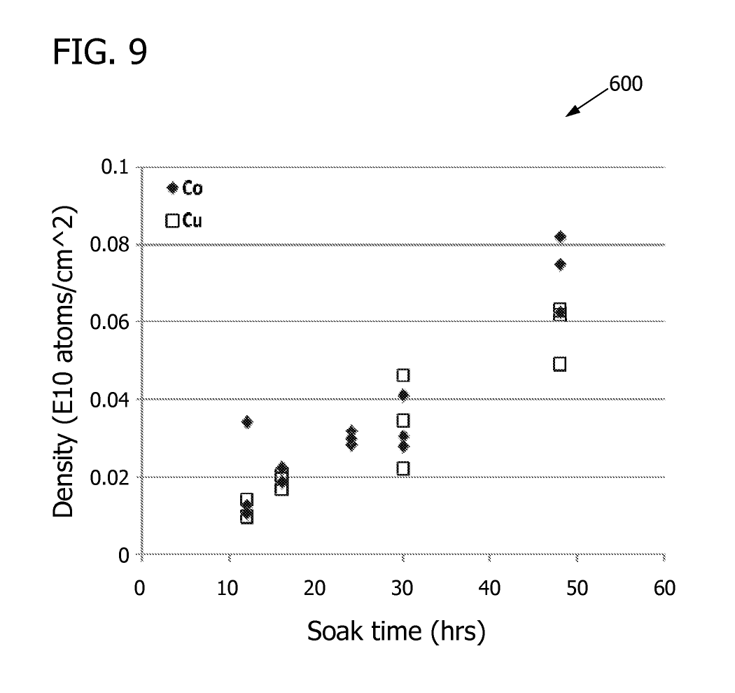

FIGS. 9-11 depict examples of experimental data in the form of graphs. The graphs generally show the density of various contaminants deposited on the surface of the wafer W at predetermined times (e.g., “soak times” or “immersion times”).

In FIG. 9, a graph 600 shows the density of cobalt and copper as a function of different soak times. As shown in the graph 600, the density of cobalt and copper on the surface of the wafer W increases in a generally linear fashion as the soak times increase.

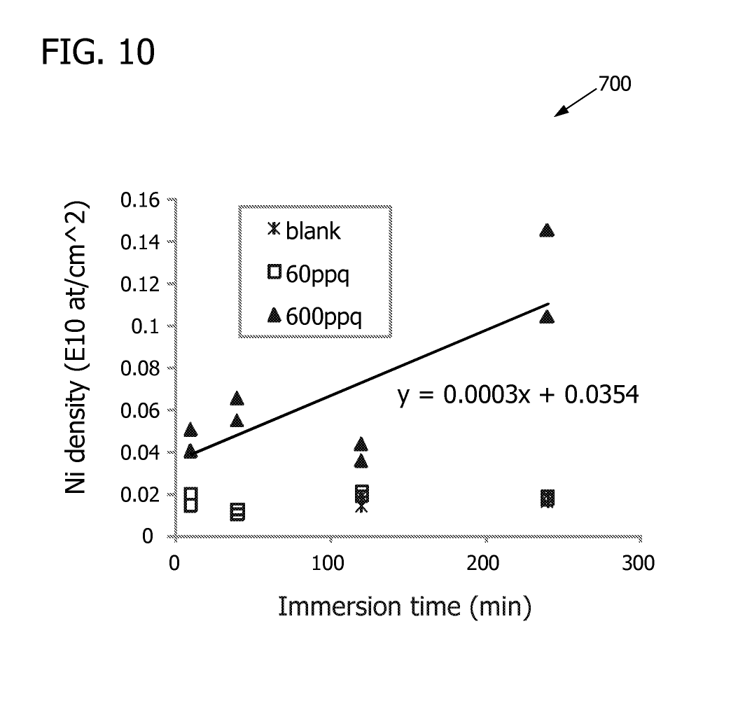

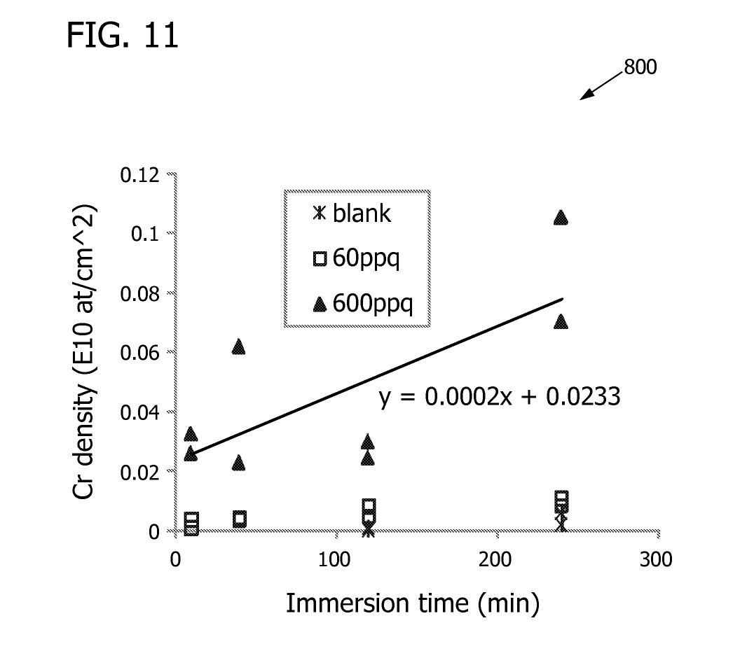

Some of the data shown in graphs 700, 800 of FIGS. 10 and 11, respectively, were obtained by “spiking” the water in which the wafers were immersed with contaminant-containing solutions. Three data points are shown for each of the different immersion times. The first data point is referred to as “blank” and represents instances where the water was not spiked with any contaminant-containing solution. However, even when the water was not spiked, relatively low background levels of contaminants were present in the water. The second data point represents instances where the water was spiked with contaminant-containing solution having 60 parts per quadrillion contaminant concentration. The third data point represents instances where the water was spiked with contaminant-containing solution having 600 parts per quadrillion contaminant concentration.

The graph 700 of FIG. 10 shows the density of nickel for three nickel-containing solutions on the surface of the wafer W as a function of different immersion times. The density of the nickel on the surface of the wafer W increases in a generally linear manner as the immersion times increase for a solution having 600 parts per quadrillion of nickel.

FIG. 11 (graph 800) shows the density of chromium for three chromium-containing solutions on the surface of the wafer W as a function of different immersion times. Again, the first data point is referred to as “blank” and represents instances where the water was not spiked with any contaminant-containing solution. As shown in the graph 800, the density of the chromium on the surface of the wafer W increases in a generally linear manner as immersion times increase for a solution having 600 parts per quadrillion of chromium.

When introducing elements of the present invention or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description and shown in the accompanying drawing[s] shall be interpreted as illustrative and not in a limiting sense.

What is claimed is:

1. A method for determining metal content in a container of water, the method comprising;
contacting a substrate with the water for a predetermined period of time, the substrate having a surface;
drying the substrate;
analyzing the substrate surface to determine metal content; and
determining the metal content in the water from the metal content on the substrate surfaces.

2. The method of claim 1 wherein the predetermined period of time is at least 500 minutes.

3. The method of claim 1 wherein the predetermined period of time is at least 700 minutes.

4. The method of claim 1 wherein the predetermined period of time is 750 minutes.

5. The method of claim 1 wherein the metal content includes nickel.

6. The method of claim 1 wherein contacting the substrate with the water comprises immersing the substrate in water.

7. The method of claim 1 wherein contacting the substrate with the water comprises directing a flow of water to contact the substrate surface.

8. The method of claim 1 wherein the substrate is a semiconductor wafer.

9. The method of claim 1 further comprising analyzing the substrate surface to determine metal content on the substrate surface prior to contacting the substrate with the water.

10. The method of claim 1 further comprising placing the substrate in the container while the water is present in the container.

11. The method of claim 10 wherein the substrate is oriented substantially parallel to a vertical sidewall of the container.

12. The method of claim 10 wherein the substrate is oriented substantially perpendicular to a vertical sidewall of the container.

13. The method of claim 10 wherein placing the substrate in the container comprises placing the substrate in a support member.

14. The method of claim 13 wherein placing the substrate in a support member comprises placing the substrate in the support member using one of a vacuum wand and a robotic effector.

15. The method of claim 13 wherein the support member is formed from a material that does not release contaminants when in the presence of the water.

16. The method of claim 13 wherein the support member is coated with a layer of material that is non-reactive with the water.

17. The method of claim 10 further comprising cleaning the container prior to placing the substrate in the container.

18. The method of claim 17 wherein cleaning the container comprises washing the container with acid.
